(19) United States
(12) Patent Application Publication
(10) Pub. No.: US 2015/0182239 A1
(43) Pub. Date: Jul. 2, 2015

(54) ENDOSCOPIC FIXATION OF A MEDICAL DEVICE USING MUCOSAL CAPTURE

(71) Applicant: BFKW, LLC, Grand Rapids, MI (US)

(72) Inventors: Randal S. Baker, Ada, MI (US); Frederick J. Walburn, Grand Rapids, MI (US)

(21) Appl. No.: 14/572,230

(22) Filed: Dec. 16, 2014

Related U.S. Application Data
(60) Provisional application No. 61/923,050, filed on Jan. 2, 2014.
(60) Provisional application No. 61/951,088, filed on Mar. 11, 2014.

(51) Int. Cl. A61B 17/22; A61B 1/04

(52) U.S. Cl. CPC A61B 17/22031; A61B 1/04; A61B 2017/22034; A61B 2017/22074

(57) ABSTRACT

An endoscopic method of fixing an intraluminal device in a lumen or hollow organ lined with mucosa includes positioning the intraluminal device in the lumen or hollow organ lined with mucosa and having a fixation device at a distal end portion of the endoscope. A fixation device at a distal end of an endoscope includes a body having a distal sealing portion. The sealing portion is positioned against the wall of the intraluminal device over a mucosal capture opening. Suction is applied through the endoscope and body to at least partially draw mucosa into the mucosal capture opening. The mucosa is retained in the mucosal capture opening.

### CROSS REFERENCE TO RELATED APPLICATIONS

This application claims the priority benefits of U.S. patent application Ser. No. 61/923,050, filed on Jan. 2, 2014, and U.S. patent application Ser. No. 61/951,088, filed on Mar. 11, 2014, the disclosures of which are hereby incorporated herein by reference in their entirety.

### BACKGROUND OF THE INVENTION

The present invention is directed to a method and device for fixation of a medical device in a lumen or hollow organ lined with mucosa and, in particular, to an endoscopic technique.

In our U.S. Pat. No. 8,894,670 issued Nov. 25, 2014, entitled MUCOSAL CAPTURE FIXATION OF MEDICAL DEVICE, we disclose a technique for fixing a luminal device in a lumen or hollow organ lined with mucosa against shear forces caused by peristaltic waves tending to cause distal migration. Mucosal capture openings are provided in a wall of the luminal device, and the wall of the luminal device is caused to expand outwardly which tends to cause mucosa to budge into the mucosal capture openings. A retainer may be provided to retain the section of the mucosa within the mucosal capture opening. A vacuum may be applied to assist in capturing the mucosa.

### SUMMARY OF THE INVENTION

An endoscopic fixation device that is adapted to fix an intraluminal device in a lumen or hollow organ lined with mucosa, according to an aspect of the invention, includes a body having a distal sealing portion. The sealing portion defines a distal surface configured to form a fluid seal with a wall of the intraluminal device. The body defining a suction passage to communicate suction from the endoscope to the distal surface to at least in part draw mucosa into the mucosal capture opening.

The fixation device may include a support portion that has sufficient strength to support the body at a distal end of an endoscope. The support portion may be generally rigid. The sealing portion may be pliant. The distal surface may be at an angle to the axis of elongation of the endoscope. The angle may be between approximately 30 degrees and approximately 60 degrees and may be about 45 degrees.

The body may provide a generally unobstructed visual passage to visualize the mucosal capture opening. The suction passage may be defined by a generally tubular member with the visual passage extending through the generally tubular opening. The suction passage may be defined by a generally tubular member with the visual passage being around the tubular member. The generally tubular member may be supported at the suction channel of the endoscope and the visualization channel of the endoscope being outside of the tubular member to define the visual passage. The generally tubular member may form an interference fit with the suction channel of the endoscope.

The fixation device may include a band dispenser that is configured to dispense at least one band around mucosa extending through the mucosal capture opening. The band dispenser may be configured to dispense multiple bands, each around mucosa extending through a different mucosal capture opening. The band dispenser may be operable through a channel in the endoscope. The band dispenser may include a band support area. The band support area may be at an interface with the distal end portion of the endoscope.

The fixation device may be combined with one or more bands that are configured to maintain perfusion in a mucosa extending through the mucosal capture opening. The band(s) may have a generally circular cross section. The band(s) may have a cross section with an enlarged portion in order to resist passing of the band through the mucosal capture opening when engaged with mucosa extending through that mucosal capture opening. The fixation device may be combined with a loop application device that is configured to apply a loop around a mucosa extending through the mucosal capture opening.

A method of fixing an intraluminal device in a lumen or hollow organ lined with mucosa, the intraluminal device having a wall and at least one mucosal capture opening in the wall, according to an aspect of the invention, includes positioning the intraluminal device in the lumen or hollow organ lined with mucosa. A fixation device with a distal sealing portion is positioned with the sealing portion against the wall of the intraluminal device generally around the at least one mucosal capture opening. Suction is applied through the fixation device positioned against the wall of the intraluminal device to at least partially draw mucosa into the mucosal capture opening. The mucosa is retained in the mucosal capture opening.

An endoscopic method of fixing an intraluminal device in a lumen or hollow organ lined with mucosa, according to an aspect of the invention, includes positioning the intraluminal device in the lumen or hollow organ lined with mucosa and having a fixation device at a distal end portion of the endoscope. The fixation device includes a body having a distal sealing portion. The sealing portion is positioned against the wall of the intraluminal device generally around a mucosal capture opening. Suction is applied through the endoscope and body to at least partially draw mucosa into the mucosal capture opening. Mucosa is retained in the mucosal capture opening.

The body of the fixation device may have a proximal support portion, such as a generally rigid portion, that has sufficient strength to support the body. The support portion may be attached to the endoscope. The body may define a suction passage in communication with a suction channel of the endoscope to apply suction to the mucosal capture opening. The body may be formed so as to not interfere with visualization of the mucosal capture opening with a visualization channel of the endoscope. The body may define a visual passage along the long axis of the endoscope to visualize the mucosal polyp or the body may be configured to define a visual passage around the body.

The sealing portion may define a distal surface that is at an angle to an axis of the endoscope. The angle may be between approximately 30 degrees and approximately 60 degrees, such as an angle of about 45 degrees. The retaining of the mucosa may include applying a band or loop around the mucosa extending into the mucosal capture opening. A band dispenser may be used to dispense a band and applying the band around mucosa in a mucosa capture opening. The band dispenser may be adapted to dispense multiple bands, each band around mucosa in a different mucosa capture opening. The band dispenser may operate through a channel in the endoscope.

The support portion may have a proximal portion that overlies the distal end portion of the endoscope and the band dispenser dispenses bands from the proximal portion of the rigid portion.

The band or loop may be applied while maintaining perfusion of the mucosa in the mucosal capture opening. The band or loop may be a band with a generally circular cross section. The band or loop may be a band having a non-circular cross section, such as with an enlarged portion in order to resist passing through the mucosal capture opening. Alternatively, the band or loop may be a loop around mucosa in the mucosal capture opening.

These and other objects, advantages and features of this invention will become apparent upon review of the following specification in conjunction with the drawings.

### BRIEF DESCRIPTION OF THE DRAWINGS

FIG. 1 is a perspective view of an endoscopic method, according to an embodiment of the invention, used to fix an intraluminal device in a lumen or hollow organ;

FIG. 2 is a perspective view of a fixation device;

FIG. 3 is the same view as FIG. 2 of an alternative embodiment thereof;

FIG. 4 is the same view as FIG. 2 of another alternative embodiment thereof;

FIG. 5 is a perspective view of a band;

FIG. 6 is a perspective view of another alternative embodiment of a fixation device;

FIG. 7 is a perspective view of another alternative embodiment of a fixation device;

FIG. 8a is a perspective view of another alternative embodiment of a fixation device connected with an end portion of an endoscope;

FIGS. 8b and 8c are the same view as FIG. 8a of the fixation device per se;

FIG. 9a is a perspective view of another alternative embodiment of a fixation device connected with an end portion of an endoscope;

FIG. 9b is the same view as FIG. 9a of the fixation device per se;

FIG. 10 is a perspective view of another alternative embodiment of a fixation device;

FIG. 11 is a perspective view of another alternative embodiment of a fixation device;

FIG. 12 is a perspective view of another alternative embodiment of a fixation device;

FIG. 13 is a perspective view of another alternative embodiment of a fixation device; and

FIG. 14 is a perspective view of another alternative embodiment of a fixation device.

### DESCRIPTION OF THE PREFERRED EMBODIMENT The present embodiments implement the techniques for mucosal capture fixation disclosed in commonly assigned U.S. Pat. No. 8,894,670 issued Nov. 25, 2014 entitled MUCOSAL CAPTURE FIXATION OF MEDICAL DEVICE, the disclosure of which is hereby incorporated herein by reference.

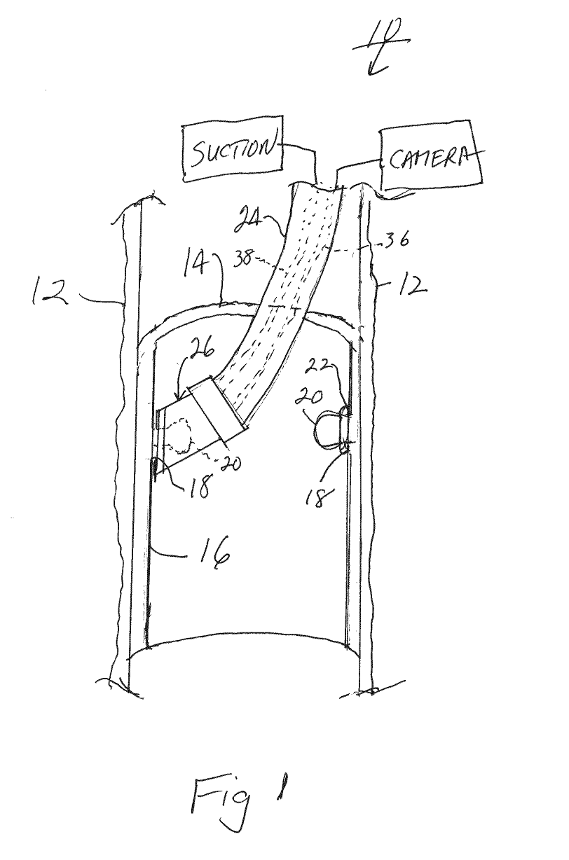

Referring now to the drawings and the illustrative embodiments depicted therein, an endoscopic method 10 is useful to fix an intraluminal device 14 in a lumen or hollow organ 12 of a human or animal body that is lined with mucosa (FIG. 1). Such lumen or hollow organ typically experiences peristalsis which tends to cause distal migration of intraluminal device 14 from the peristaltic waves travelling along the lumen or hollow organ. Examples of such lumen or hollow organ include the various segments making up the alimentary or gastrointestinal tract. However, other examples are known in the art. Intraluminal device 14 may be an esophageal or cardiac portion of a bariatric device of the type disclosed in commonly assigned U.S. Pat. Nos. 8,529,431; 8,100,931; and 7,846,174, the disclosures of which are hereby incorporated herein by reference. Alternatively, intraluminal device 14 may be an esophageal stent, a colorectal stent, or the like.

Intraluminal device 14 has a wall 16 that generally conforms to the size any shape of lumen 12 and one or more mucosal capture openings 18 that are adapted to receive and retain mucosa 20 extending into the mucosal capture opening in order to fix intraluminal device 14 in lumen 12. Such fixation will be discussed in more detail below. The mucosa 20 may be retained in opening 18 by a retainer band or loop 22, or the like, such as that is applied around the mucosa 20 that extends past the mucosal capture opening in order to resist the mucosa from being pulled back through mucosal capture openings 18. Retainer 22 may alternatively be a clip or other pressure component, such as disclosed in U.S. Pat. No. 8,721,528 for an ENDOSCOPE CAP, the disclosure of which is hereby incorporated herein by reference. Retainer 22 is tight enough to prevent polyp 20 from pulling back through openings 18, but not so tight as to cut off perfusion to the mucosal tissue extending into the opening. Retainer 22 may alternatively be a penetrating member that penetrates the mucosa 20. The use of mucosa 20 extending into mucosal capture opening(s) 18 is for the purpose of providing at least temporary fixation of device 14 sufficiently to allow tissue ingrowth to occur in tissue ingrowth openings (not shown) or even in openings 18, as disclosed in commonly assigned U.S. Pat. No. 8,672,831 issued Mar. 18, 2014, entitled BARIATRIC DEVICE AND METHOD, the disclosure of which is hereby incorporated by reference. After tissue ingrowth occurs, in a week or thereabout, the mucosa 20 drawn into mucosal capture openings 18 can slough off with the tissue ingrowth providing a more permanent, or semi-permanent, fixation of the intraluminal device in the lumen or hollow organ. However, because the capture mucosa 20 is retained by retainer 22 in a manner that maintains perfusion of the mucosa, the captured mucosa may, alternatively, provide semi-permanent fixation for longer, if needed, to accommodate temporary placement of intraluminal device 14. Retainer 22 may be made of a resorbable material that is dissolved over time. It should be understood that, in addition to mucosa, some submucosa or even muscularis may be captures in mucosa capture opening 18, but the mucosa is easiest to remove using conventional techniques, such as chemical or electrical ablation or even mechanical force in order to remove intraluminal device 14. In this manner, the intraluminal device can be securely fixed in the lumen or hollow organ but be easily removed without perforating the lumen or hollow organ or otherwise causing significant damage to the tissue thereof. Also, once the mucosa is removed, the submucosa and muscularis should not be sufficient to impede removal of device 14.

As disclosed in commonly assigned U.S. Pat. No. 8,894,670 issued Nov. 25, 2014, entitled MUCOSAL CAPTURE FIXATION OF MEDICAL DEVICE, the disclosure of which is hereby incorporated herein by reference, wall 16 is caused to expand outwardly either by the wall being self-expandable by an inflatable balloon, or the like. The outward expanding wall creates an interference with lumen 12 causing mucosa to bulge into mucosal capture openings 18 in order to at least partially provide fixation of intraluminal device 14. A fixation device 26 is fitted on the distal end of a conventional endoscope 24 in order to further draw mucosa 20 into the mucosal capture opening 18. This is accomplished by fixation device 26 extending a suction passage 38 of endoscope 24 to an individual mucosal capture opening 18 in order to apply suction to the mucosal capture opening. The fixation device does not interfere with a visualization passage 36 of endoscope 24 in a manner that allows the physician to visualize the mucosal capture opening. Once manipulation of the endoscope causes fixation device 26 to be positioned over the opening 18, fixation device 26 forms a seal against wall 16 of intraluminal device 14 around the mucosal capture opening 18. This seal enhances the amount of suction that can be applied to the mucosa. This facilitates the formation of mucosa 20 passing into mucosal capture opening past wall 16. Once the mucosa 20 is drawn through opening 18 past wall 16, a band or loop 22 is applied to the proud mucosa. This causes the mucosa to form an enlarged portion, or head, which tends to keep the band or loop in place. Also, the band or loop in combination with the enlarged head of mucosa 20 tends to resist the mucosa from pulling through the mucosal capture opening 18.

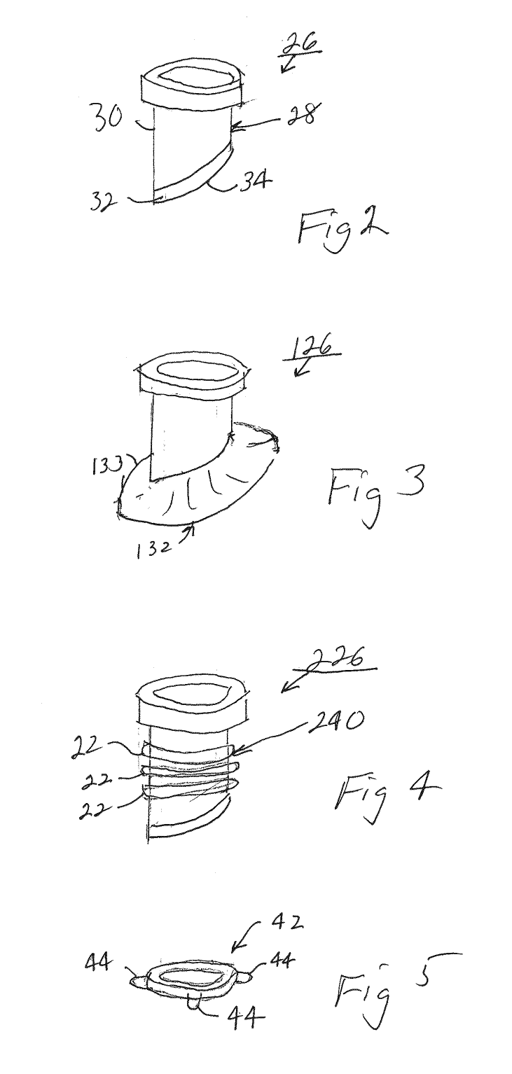

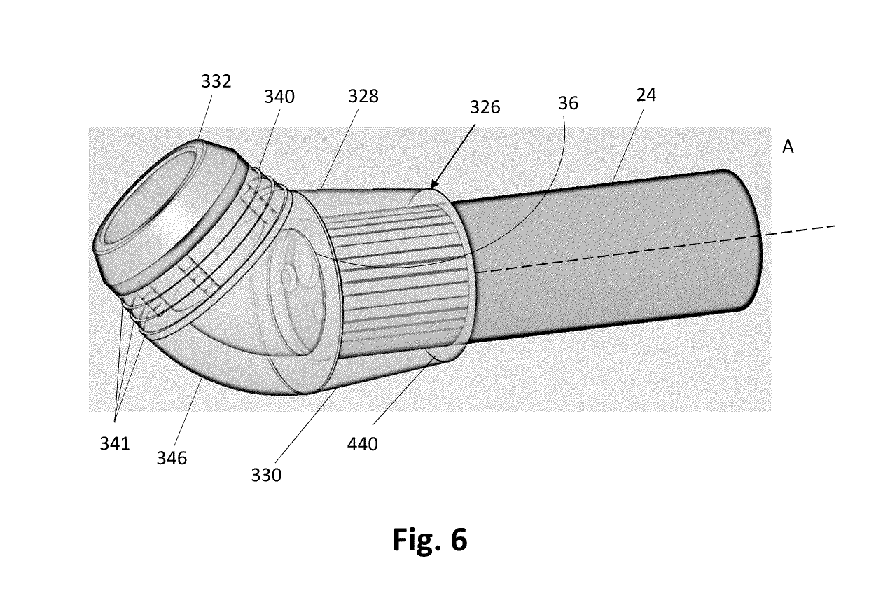

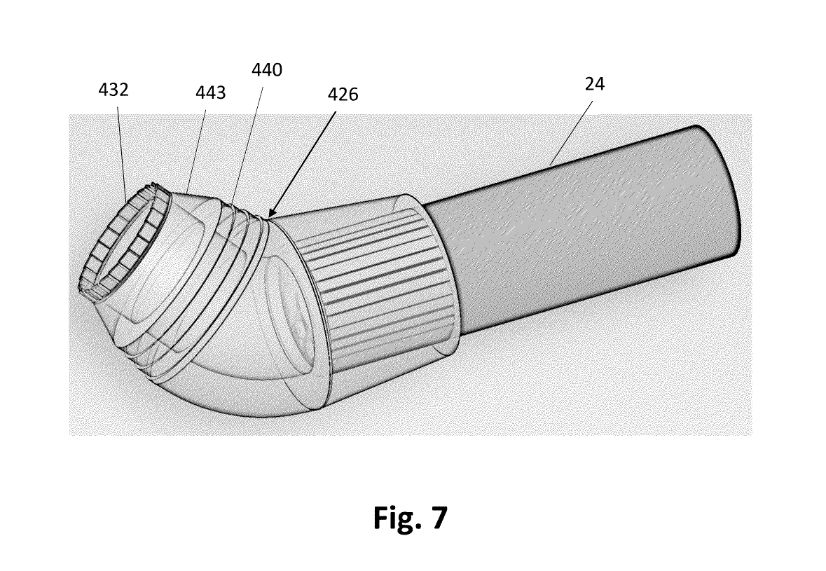

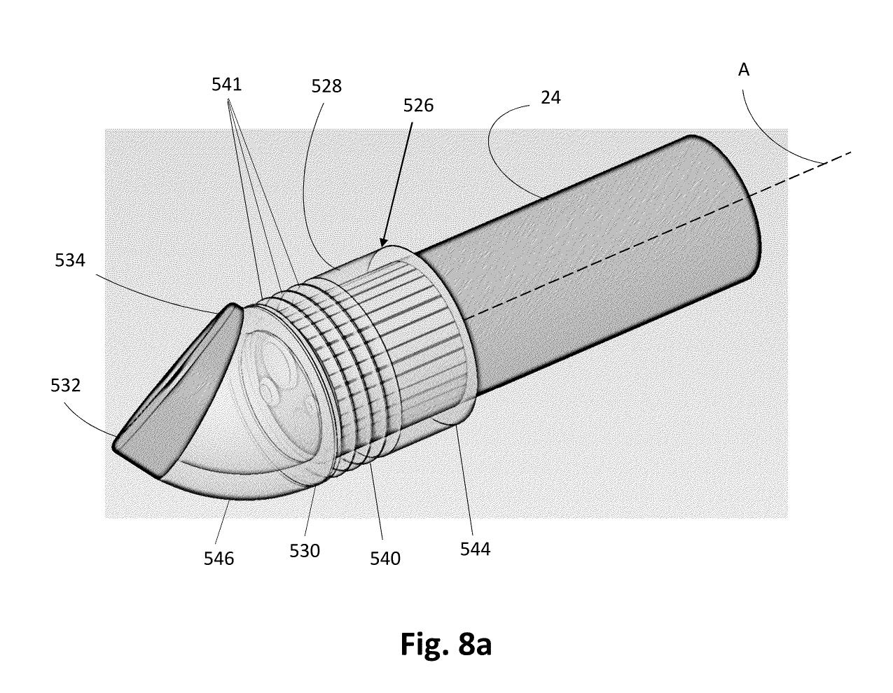

Fixation device 26 has a body 28 made up of a proximal portion 30 and a distal portion 32 both of which are open in order to extend suction passage 38 through the body. Distal portion 32 forms a seal, in the illustrated embodiment, by being pliant and defines a distal surface 34. By pliant is meant that distal surface 34 can be distorted in order to conform to the surface features of wall 16 of the interior of intraluminal device 14 and thereby establish a pneumatic seal around a mucosal capture opening 18 to enhance the suction. Distal surface 34 is at an acute angle with respect to the axis of elongation of endoscope 24 in order to allow distal surface 34 to be seated against the inner wall of intraluminal device 14. In the illustrated embodiment, distal surface 34 is at an angle to the axis of elongation of the endoscope of between about 30 degrees and about 60 degrees and is illustrated at about 45 degrees. However, body 28 allows visual passage 36 to extend to the mucosal capture opening. In the embodiment illustrated in FIG. 2, visual passage 36 extends through body 28 which would not be easily accomplished if distal surface 34 were at a significantly greater angle to the axis of elongation of endoscope 24. Endoscope 24 is capable of a limited amount of lateral flexing even within the tight interior of intraluminal device 14, as seen in FIG. 1. Thus, with a combination of the compliancy of distal portion 32 and flexing of endoscope 24, distal surface 34 can conform to the interior wall of intraluminal device 14 without interfering with visual passage 36 extending to the mucosal capture opening. In the illustrated embodiment, proximal portion 30 of body 28 is rigid or semi-rigid, also referred to as generally rigid. By generally rigid is meant that proximal portion 30 sufficiently resists deformation in order to support body 28 via a connection with endoscope 24 and to counteract the tension needed to mount and dispense bands as will be discussed in further embodiments. However, it would be possible for the entire body 28 to be made from a common material if that material were sufficiently compliant to form a seal with the wall of the intraluminal device and sufficiently rigid to support the body and mount bands.

An alternative embodiment 126 of a fixation device has a pliant distal portion 132 that has outwardly flared bell 133 that provides even further compliance with an inner surface of wall 16 of the intraluminal device around the mucosal capture openings.

An alternative embodiment 226 of a fixation device has a band-dispensing mechanism 240. Mechanism 240 allows multiple circular bands 22 to be dispensed one at a time by an actuator that is operated through a channel in the endoscope. Multiple-band-dispensing mechanisms are known in the art as disclosed in U.S. Pat. Nos. 5,398,844; 6,149,659 and 6,235,040, the disclosures of which is hereby incorporated herein by reference. The benefit of dispensing bands one at a time allows method 10 to be carried out with respect to multiple mucosal capture openings to retain mucosal in the multiple openings without the need to withdraw the fixation device and endoscope from the patient. Alternatively, the mucosa extending through the mucosal capture openings could be retained by an endo-loop device that is commercially available, particularly one that is capable of deploying and cinching a loop through the working channel of the endoscope. Such endo-loop device would be manipulated by the physician in a manner to maintain perfusion in the mucosa passing through the opening.

While conventional ligation band designs of a circular cross section may be used, a band 42 enhances the resistance of the band from being pulled through the mucosal capture opening. This is accomplished by providing a non-circular cross section, such as by enlarging the cross-sectional area of the band. For example, one or more wings 44 may be provided to the band. Other shapes may suggest themselves to the skilled artisan. For example, the band may be heat sensitive to swell in the surface area when exposed to body heat once it is positioned around the mucosa.

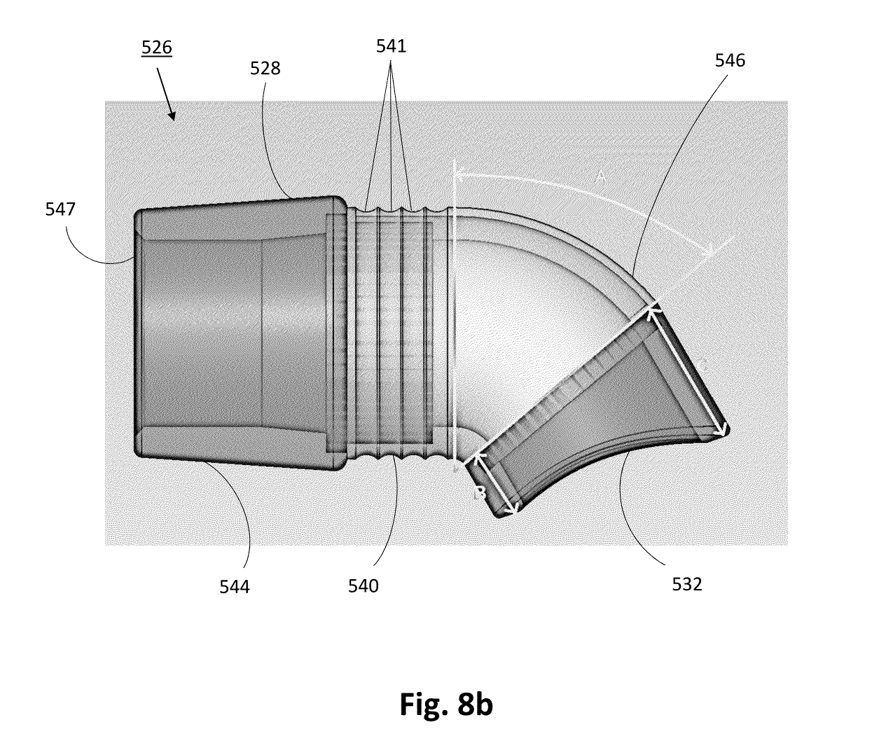

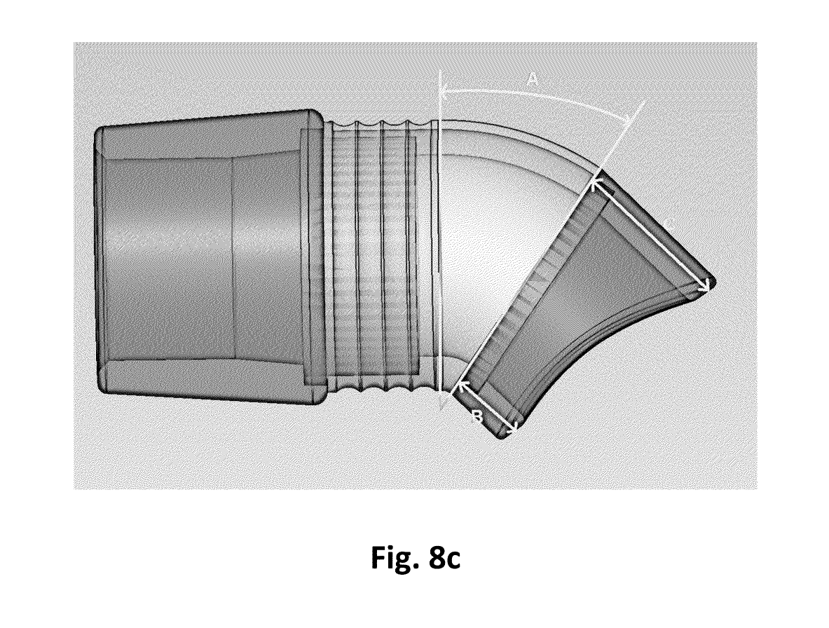

Another embodiment of a fixation device 326 is fixed to a distal end of an endoscope 24 (FIG. 6). The fixation device includes a body 328 having a rigid proximal portion 330 with a collar 344 that fits over a distal end of endoscope 24 and may be retained there by an interference fit, a frictional surface, or the like. Rigid portion 330 additionally includes an angled portion 346 that positions a seal in the form of a pliant distal portion 332 at an angle to the axis of elongation A of endoscope 24. Angled portion 346 is configured to allow visualization through distal portion 332 from visual passage 36 of the endoscope. In the illustrated embodiment, pliant surface 334 is at an angle of about 45 degrees to the axis of elongation of the endoscope. A band dispenser 340 includes holding positions 341 for multiple bands (not shown) at the distal portion of rigid portion 330 adjacent pliant portion 332. This allows the bands to be dispensed with minimal amount of travel. Pliant distal portion 332 includes a steel reinforcement scaffold (not shown) to limit deformation of the pliant portion during band dispensing as the band passes over the pliant portion.

Another embodiment of a fixation device 426 is similar to fixation device 326 except that it includes a pliant distal portion 432 that is made from a lamella material. A band dispenser 440 includes a transitional portion 443 that angles toward pliant distal portion 432 to assist in dispensing of bands.

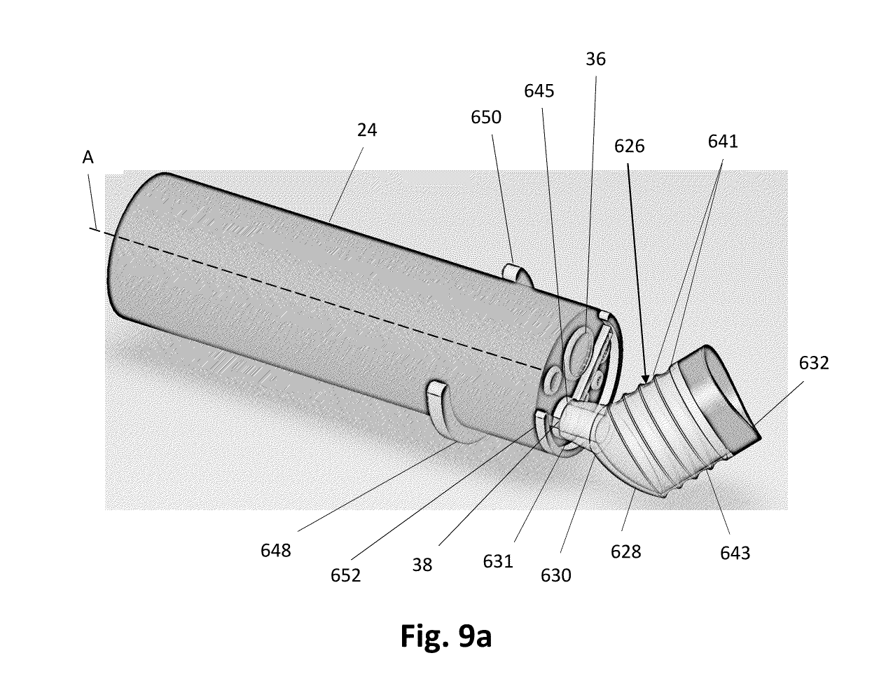

Another embodiment of a fixation device 526 includes a body 528 with a rigid portion 530 that forms a connection 544 with the endoscope in the form of a collar that extends over the distal portion of endoscope 24 and forms an interference fit, high friction fit, or the like (FIG. 8a). Fixation device 526 has a band-dispensing mechanism 540 including band-holding positions 541 that are positioned on collar 544 that overlays the end of the endoscope. An angled portion 546 terminates in a pliant distal portion 532 that forms a seal. By storing the bands at band-holding positions 541 that overlays the end of the endoscope rather than on the angled portion, angled portion 546 can be made smaller thus enhancing the ability of the visualization passage of the endoscope to pass through body 528 without partial blockage. Pliant distal portion 532 is formed as a wave-shape in order to enhance its ability to conform to the inner surface of wall 16 of the luminal device. Distal surface 534 is at an angle of about 30 degrees to the axis of elongation of the endoscope in FIG. 8a.

The collar 544 of fixation device 526 is shown in more detail in FIGS. 8b and 8c to include an engagement surface 547 that forms the interference fit with an end portion of the endoscope which extends beyond engagement surface to under band-holding positions 541. This mounting to the endoscope positions the visual passage of the endoscope as close as possible to angled portion 546. FIG. 8b shows a fixation device 526 with an angle A of the angled portion at 60 degrees. Angle A is the complementary angle to the axis of elongation of the endoscope. Thus, with angle A of the angled portion at 60 degrees, as shown in FIG. 8b, the angle of distal portion 532 to the axis of elongation of the endoscope is 30 degrees. Pliant distal portion 532 is shown varying in length from 3 to 7 mm at its shortest side B to 7 to 11 mm at its longest side C, other dimensions being possible. FIG. 8c illustrates fixation device 526 with an angle A of the angled section at 35 degrees. In the illustrated embodiment, angle A is between approximately 35 degrees and 60 degrees, although a greater or lesser angle may be provided.

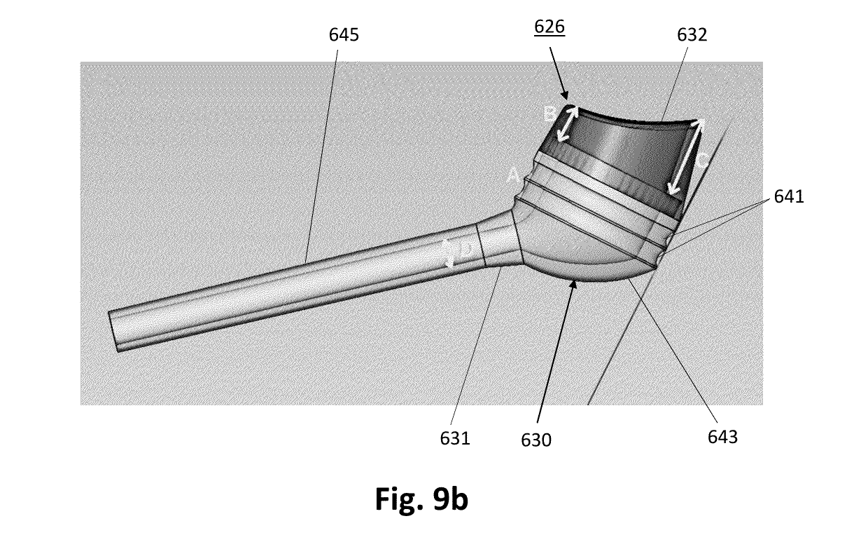

Another embodiment of a fixation device 626 includes a body 628 having a generally rigid proximal portion 630 and a seal in the form of a pliant distal portion 632 (FIG. 9a). Rigid portion 630 includes a narrow proximate portion 631 that narrows to form a connection 645 that has a sufficiently narrow diameter to fit within a distal opening of suction passage 38 of the endoscope, where it supports body 628. An optional bracket 648 retains body 628 to the endoscope. A transition portion 643 includes a band dispenser band-holding area 641 adjacent pliant distal portion 632. Configuration of body 628 defines a visualization passage from visualization passage 36 of the endoscope around the outside of body 628 to allow visualization of a mucosal capture opening of the intraluminal device. This allows the physician to align pliant distal portion 632 with the mucosal capture opening to apply suction thereto. Transition portion 643 positions pliant distal portion 632 at an angle of about 30 degrees to the long axis A of the endoscope. Optional bracket 648 provides a rigid mechanical clip 650 to form an interference fit with the endoscope and a soft clear string 652 over the end of the endoscope to engage the connection 645 of the fixation device. Connection 645 may be sufficient to mount body 628 to the endoscope without the bracket 648. Seal 632 is made up of a pliant distal portion that is wave shaped.

As seen in FIG. 9b, pliant distal portion 632 has a short side B that may vary in length from about 3 mm to about 7 mm and a long side C that may vary in length from about 5 mm to about 9 mm although greater or lesser lengths may be used. The transition between the short side B and the long side C is curved in order to define the wave shape. A diameter D of the suction channel is between about 1.4 mm and 1.7 mm, depending on the size of the suction channel of the endoscope.

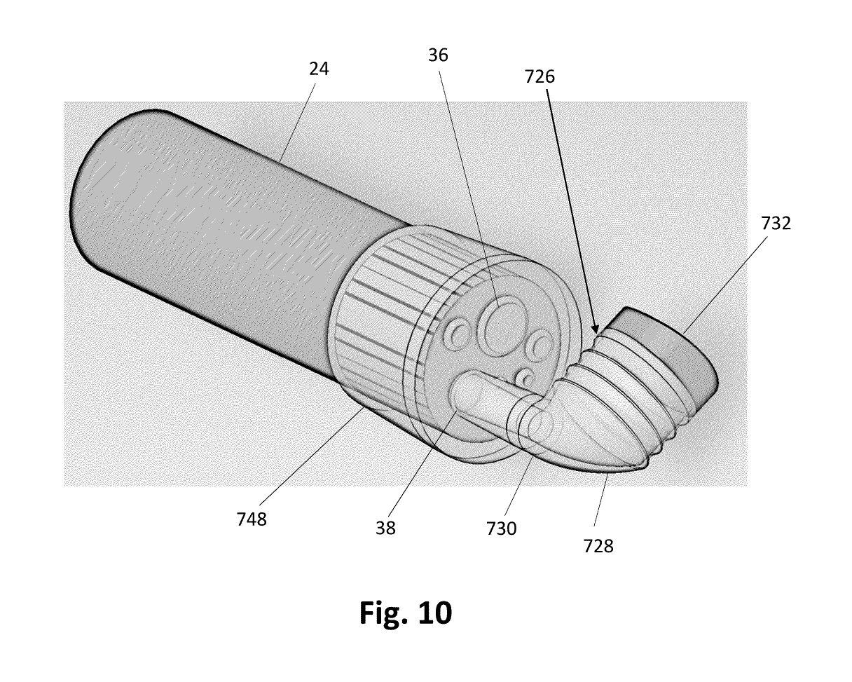

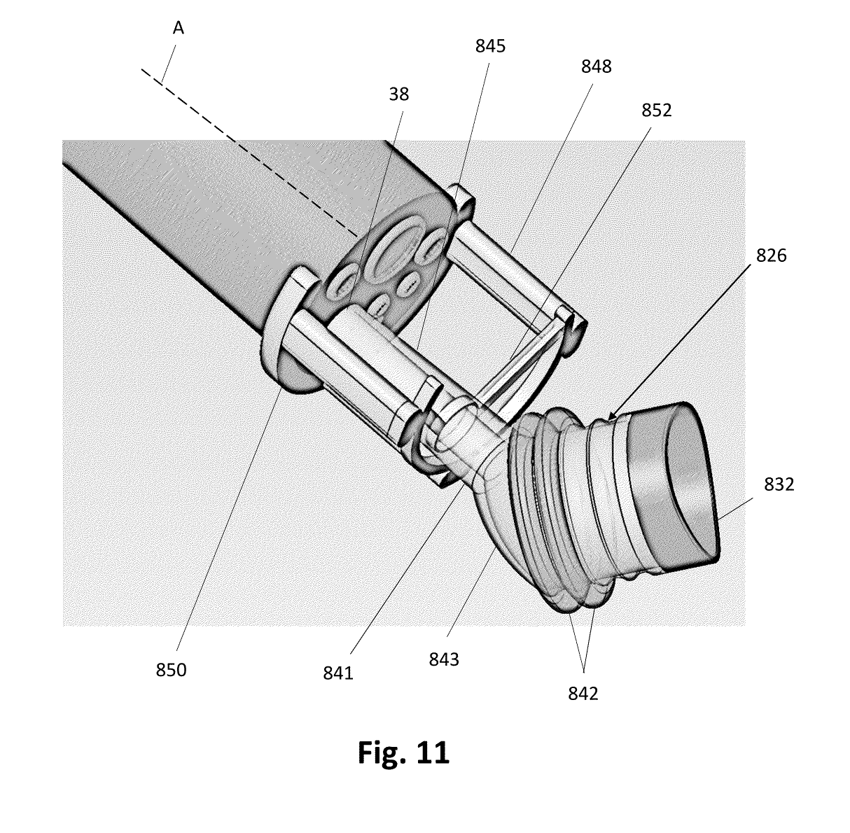

An alternative fixation device 726 includes a body 728 having a proximal portion 730 that is similar to proximal portion 630 except that it is retained to the distal end of the endoscope by a bracket 748 that is in the form of a soft clear cover over the distal end of the endoscope (FIG. 10). A transition portion 743 includes a band dispenser band-holding area 741 that supports a seal in the form of a pliant distal portion 732 that is mounted generally parallel to the long axis of the endoscope. Fixation device 726 does not block the visualization passage of the endoscope to view the mucosal capture opening and does not require the endoscope to be flexed in order to be aligned with the inner surface of the intraluminal device wall. Also, the fixation device fits generally within the cross sectional area of the endo scope thus allowing for easier manipulation of the endoscope. An alternative fixation device 826 includes a holding portion 841 that defines a connection 845 that fits within the suction passage 38 of the endoscope and a transition portion 843 that mounts the bands 842 of the band-dispensing mechanism (FIG. 11). A seal 832 is in the form of a pliant wave shape distal portion. Seal 832 is at an angle of about 30 degrees to the axis of elongation A of the endoscope. A bracket 848 includes a clip 850 that engages the endoscope and a string 852 that engages holding portion 841 of the fixation device.

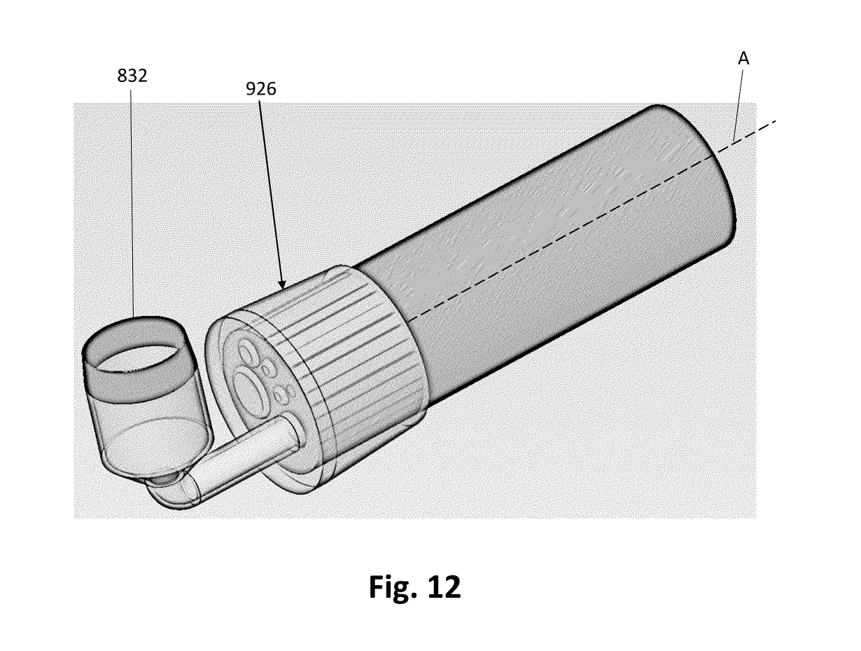

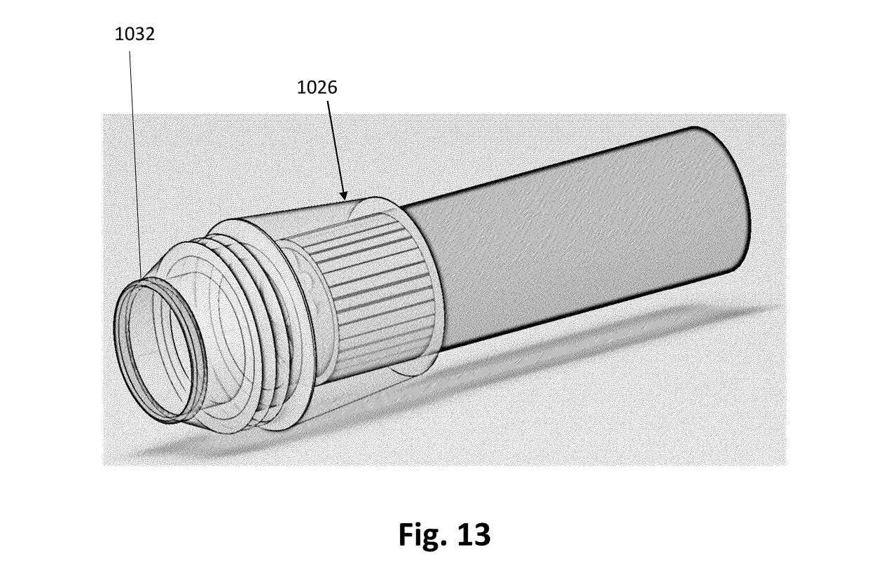

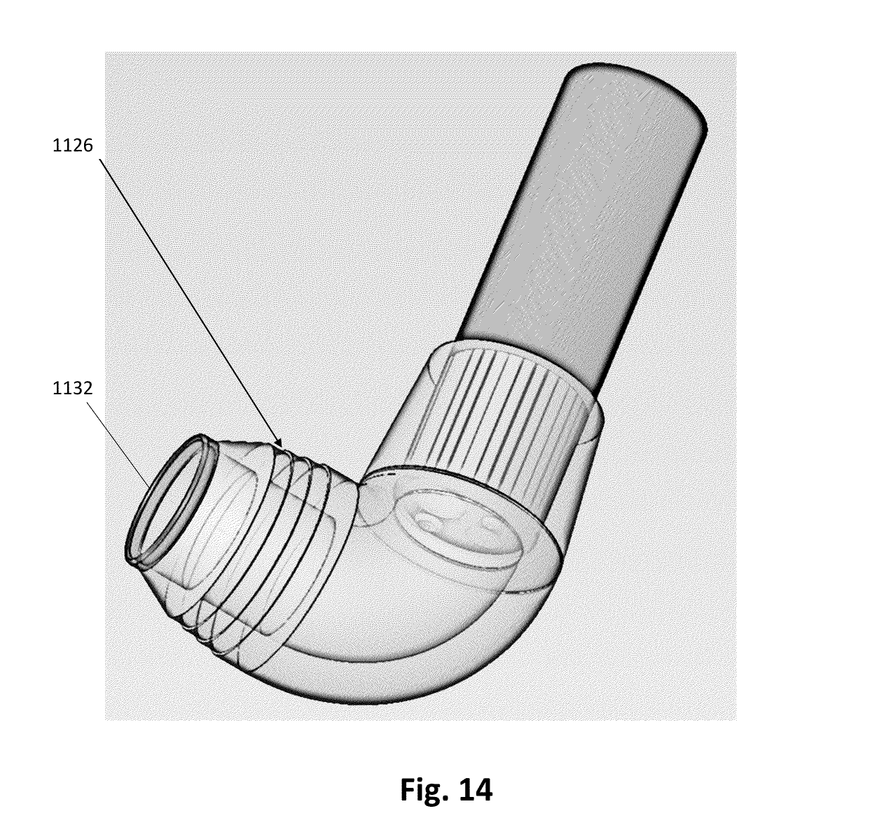

An alternative fixation device 926 is similar to fixation device 726 except without a band-dispensing mechanism (FIG. 12). Just as with fixation device 726, device 926 does not require flexing of the endoscope in order to provide a seal with the inner surface of the intraluminal device wall as a seal 832 is parallel an axis of elongation A of the endoscope. An alternative fixation device 1026 is similar to fixation device 326 except that the distal sealing portion 1032 is generally perpendicular to the axis of elongation of the endoscope (FIG. 13). An alternative fixation device 1126 is similar to fixation device 326 except that the distal sealing portion 1132 is generally parallel to the axis of elongation of the endoscope (FIG. 14).

While the foregoing description describes several embodiments of the present invention, it will be understood by those skilled in the art that variations and modifications to these embodiments may be made without departing from the spirit and scope of the invention, as defined in the claims below. The present invention encompasses all combinations of various embodiments or aspects of the invention described herein. It is understood that any and all embodiments of the present invention may be taken in conjunction with any other embodiment to describe additional embodiments of the present invention. Furthermore, any elements of an embodiment may be combined with any and all other elements of any of the embodiments to describe additional embodiments.

The embodiments of the invention in which an exclusive property or privilege is claimed are defined as follows:

1. An endoscopic intraluminal device fixation device that is adapted to fix an intraluminal device in a lumen or hollow organ lined with mucosa, said intraluminal device having a device wall and at least one mucosal capture opening in said wall, said endoscopic intraluminal device fixation device comprising:
a body having a proximal support portion and a distal sealing portion defining a distal surface;
said support portion has sufficient strength to support said body from a distal end of an endoscope and said sealing portion configured to form a fluid seal with the wall of the intraluminal device; and
said body defining a suction passage, said suction passage adapted to communicate suction from the endoscope to said distal surface to at least partially draw mucosa into the mucosal capture opening.

2. The fixation device as claimed in claim 1 wherein said distal surface is at an angle to an axis of the endoscope.

3. The fixation device as claimed in claim 2 wherein said angle is between approximately 30 degrees and approximately 60 degrees.

4. The fixation device as claimed in claim 1 wherein said body provides a generally unobstructed visual passage to visualize the mucosal capture opening.

5. The fixation device as claimed in claim 4 wherein said suction passage is defined by a generally tubular member and wherein said visual passage is through said generally tubular opening.

6. The fixation device as claimed in claim 1 including a retainer dispenser that is adapted to dispense at least one retainer around mucosa in the mucosal capture opening.

7. The fixation device as claimed in claim 6 wherein said retainer dispenser is adapted to dispense multiple retainers, each around a mucosa extending through a different mucosal capture opening.

8. The fixation device as claimed in claim 6 wherein said retainer dispenser comprises a band support area wherein said body has an interface portion that is adapted to engage a distal end portion of the endoscope and wherein said band support area is at said interface portion.

9. The fixation device as claimed in claim 6 in combination with at least one retainer that is adapted to maintain perfusion in mucosa in the mucosal capture opening.

10. The fixation device as claimed in claim 9 wherein the at least one retainer has a cross section with an enlarged portion in order to resist passing of the band through the mucosal capture opening.

11. The fixation device as claimed in claim 1 wherein said proximal portion is generally rigid and said distal sealing portion is pliant.

12. An endoscopic method of fixing an intraluminal device in a lumen or hollow organ lined with mucosa, said intraluminal device having a wall and at least one mucosal capture opening in said wall, said endoscopic method comprising:
positioning the intraluminal device in the lumen or hollow organ lined with mucosa;
having a fixation device at a distal end portion of the endoscope, said fixation device comprising a body having a distal sealing portion;
positioning said sealing portion against the wall of the intraluminal device generally around said at least one mucosal capture opening;
applying suction through the endoscope and body positioned against the wall of the intraluminal device to at least partially draw mucosa into the mucosal capture opening; and
retaining the mucosa in the mucosal capture opening.

13. The method as claimed in claim 12 wherein said body defining a suction passage in communication with a suction channel of the endoscope to apply suction to the mucosal capture opening.

14. The method as claimed in claim 12 wherein said body does not interfere with visualization of the mucosal capture opening with a visualization channel of the endoscope.

15. The method as claimed in claim 14 wherein said body defines a visual passage.

16. The method as claimed in claim 12 wherein said sealing portion is compliant.

17. The method as claimed in claim 12 wherein said sealing portion defines a distal surface that is at an angle to an axis of the endoscope, said angle being between approximately 30 degrees and approximately 65 degrees.

18. The method as claimed in claim 12 including a retainer dispenser that is adapted to dispense a retainer and wherein said retaining includes applying a retainer with said retainer dispenser around a mucosa in the mucosa capture opening.

19. The method as claimed in claim 18 wherein said retainer dispenser is adapted to dispense multiple bands, and wherein said applying a band includes applying each band around mucosa extending in a different mucosal capture opening.

20. The method as claimed in claim 12 wherein said body includes a generally rigid proximal portion wherein said rigid portion has a proximal portion that overlies the distal end portion of the endoscope.

21. The method as claimed in claim 12 including maintaining perfusion of mucosa in the mucosal capture opening.

22. A method of fixing an intraluminal device in a lumen or hollow organ lined with mucosa, said intraluminal device having a wall and at least one mucosal capture opening in said wall, said method comprising:
positioning the intraluminal device in the lumen or hollow organ lined with mucosa;
having a fixation device with a sealing portion;
positioning said sealing portion against the wall of the intraluminal device generally around said at least one mucosal capture opening;
applying suction through the fixation device positioned against the wall of the intraluminal device to at least partially draw mucosa into the mucosal capture opening; and
retaining the mucosa in the mucosal capture opening.
